(19) United States
(12) Patent Application Publication
(10) Pub. No.: US 2020/0088723 A1
(43) Pub. Date: Mar. 19, 2020

(54) DIFFERENTIAL SENSOR MEASUREMENT METHODS AND DEVICES

(71) Applicant: FemtoDx, Beverly Hills, CA (US)

(72) Inventors: Pritiraj Mohanty, Los Angeles, CA (US); Shyamsunder Erramilli, Quincy, MA (US)

(73) Assignee: FemtoDx, Beverly Hills, CA (US)

(21) Appl. No.: 16/211,199

(22) Filed: Dec. 5, 2018

Related U.S. Application Data
(63) Continuation-in-part of application No. 14/510,178, filed on Oct. 9, 2014, now abandoned.
(60) Provisional application No. 62/594,729, filed on Dec. 5, 2017.
(60) Provisional application No. 61/888,830, filed on Oct. 9, 2013.

(51) Int. Cl. G01N 33/543; G01N 27/22; G01N 27/12

(52) U.S. Cl. CPC G01N 33/54373; G01N 27/125; G01N 27/227

(57) ABSTRACT

Semiconductor-based sensor devices and methods for detection of biological agents are described herein.

### RELATED APPLICATIONS

This application claims priority to U.S. Provisional Application Ser. No. 62/594,729, filed Dec. 5, 2017, which is incorporated herein by reference in its entirety. This application is also a continuation-in-part (CIP) of U.S. patent application Ser. No. 14/510,178, filed Oct. 9, 2014 which claims priority under 35 U.S.C. § 119(e) to U.S. Provisional Patent Application Ser. No. 61/888,830, filed Oct. 9, 2013, which are both incorporated herein by reference in their entireties.

### BACKGROUND

Biosensors generally comprise devices integrated with a binding molecule (also referred to herein as a “detector molecule”) and a signal transducer, which can provide a recognition signal of the presence of a specific target (e.g., an analyte). Biosensors can be based on any number of physical principles, but generally depend on binding of a binding molecule to a target analyte. The specific binding or reaction between the binding molecule and the analyte can introduce a signal that is then transduced and measured. Biosensors can be configured for macromolecular recognition, such as with human cells of different types, viruses, and pathogenic organisms. Therefore, there is a far-reaching diagnostic utility in these devices ranging from applications towards human health, food safety, drug response, and personalized medicine.

One major challenge to biosensor development is signal noise. Some sensors, for example, are connected to external measurement devices such as ohmmeters, voltmeters, and ammeters, for the purposes of detecting resistance changes. In some such cases, the sensors are in the middle of a large fluid volume, and because connections must be made outside of the fluid, very long electrodes, cables, and other circuitry are used. As a result, the electrodes and other components may suffer from very large contact resistance when coupled to the sensor. All of these factors contribute to significant noise and background in any measurement. Other sources of noise may be related to induction loops, in the circuitry, noise generated by the fluid in regions where the electrodes are in contact with the fluid, thermal noise, as well as other sources. This noise, which may depend roughly on the total length of the circuit and quality of all contacts, can be much larger than any resistance change induced by analyte binding. In many cases, the noise and background are due to large-scale, external fluctuations, and therefore is the same in each segment of the surface of the sensor. Therefore, a method of eliminating such systematic noise from a measurement is highly desirable for enhanced biosensors.

### SUMMARY

Semiconductor-based sensor devices and methods for detection of biological agents are described herein.

In one aspect, a device is provided. The device comprises a first semiconductor-based sensor comprising a first insulating layer functionalized with a detector molecule. The device further comprises a second semiconductor-based sensor in electrical communication with the first semiconductor-based sensor. The second semiconductor-based sensor comprises a second insulating layer. The first insulating layer and the second insulating layer are configured such that when the first semiconductor-based sensor and the second semiconductor-based sensor are exposed to a fluid comprising an analyte, the change in an electrical property of the first semiconductor-based sensor is greater than the change in a corresponding electrical property of the second semiconductor-based sensor.

In another aspect, a device is provided. The device comprises a first semiconductor-based sensor comprising a first insulating layer functionalized with a detector molecule. The device further comprises a second semiconductor-based sensor in electrical communication with the first semiconductor-based sensor. The second semiconductor-based sensor comprises a second insulating layer. The first insulating layer is at least 2 times the thickness of the second insulating layer.

In another aspect, a method is described. The method comprises exposing a device to a fluid comprising an analyte. The device comprises a first semiconductor-based sensor comprising a first insulating layer functionalized with a detector molecule. The device further comprises a second semiconductor-based sensor in electrical communication with the first semiconductor-based sensor. The second semiconductor-based sensor comprising a second insulating layer. The method further comprises measuring a change in an electrical property of the first semiconductor-based sensor and a change in a corresponding electrical property of the second semiconductor-based sensor to determine a differential electrical property based at least in part on the change in the electrical property of the first semiconductor-based sensor and the change in the electrical property of the second semiconductor-based sensor.

In some embodiments, the second insulating layer is functionalized with the detector molecule.

In some cases the change in the electrical property of the first semiconductor-based sensor is at least two times greater than the change in a corresponding electrical property of the second semiconductor-based sensor.

In some embodiments, the capacitance between the first semiconductor-based sensor and a layer of charge formed when the first semiconductor-based sensor is exposed to the fluid and the analyte binds the detector molecule of the first semiconductor-based sensor is greater than the capacitance between the second semiconductor and a layer of charged formed when the second semiconductor-based sensor is exposed to the fluid and the analyte binds the detector molecule of the second semiconductor-based sensor.

In some cases, the thickness of the second insulating layer is greater than or equal to 2 times the thickness of the first insulating layer and less and less than or equal to 50 times the thickness of the first insulating layer.

In some embodiments, thickness of the first insulating layer is greater than or equal to 2 nm and less than or equal to 5 nm. In some cases, the thickness of the second insulating layer is greater than or equal to 10 nm and less than or equal to 100 nm.

In some embodiments, the dielectric constant of the first insulating layer is greater than the dielectric constant of the second insulating layer.

In some instances, the first insulating layer and/or the second insulating layer comprises an oxide.

In some embodiments, the detector molecule comprises an antibody, a DNA fragment, and/or RNA fragment. In some cases, the detector molecule comprises an enzyme. In certain embodiments, the detector molecule comprises glucose oxidase. In certain embodiments, the detector molecule comprises urease.

In some embodiments, the analyte comprises a biomolecule. In some cases, the analyte comprises a monosaccharide and/or a polysaccharide. In some instances, the analyte comprises glucose.

In some embodiments, the analyte comprises urea.

In some embodiments, the electrical property is an electrical property selected from the group consisting of voltage, resistance, conductance, and current.

In some cases, the first semiconductor-based sensor and/or the second semiconductor-based sensor comprises one or more nanowires.

In some embodiments, the first semiconductor-based sensor is at least partially covered by the first insulating layer, and/or the second semiconductor-based sensor is at least partially covered by the second insulating layer.

In some embodiments, the first semiconductor-based sensor and/or the second semiconductor-based sensor comprises a field effect transistor.

In some cases, the device is configured to output a differential electrical property based at least in part on the change in electrical property of the first semiconductor-based sensor and the change in electrical property of the second semiconductor-based sensor.

In some embodiments, methods comprising detecting the presence of the analyte based at least in part on the differential electrical property.

In some embodiments, the differential electrical property is determined by subtracting the change in the electrical property of the second semiconductor-based sensor from the change in the electrical property of the first semiconductor-based sensor.

Other aspects and embodiments will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1B show schematic illustrations of an exemplary semiconductor sensor in the absence (FIG. 1A) and presence (FIG. 1B) of binding of an analyte to a detector molecule, according to certain embodiments;

FIGS. 2A-2B show schematic depictions of devices comprising a first semiconductor sensor and a second semiconductor sensor, according to certain embodiments;

FIG. 3 shows a schematic depiction of devices comprising a first semiconductor sensor comprising a first insulating layer and a second semiconductor sensor comprising a second insulating layer, according to certain embodiments;

FIG. 4A depicts an equivalent circuit diagrams for the a two-point measurement;

FIG. 4B shows an equivalent circuit diagram for a device comprising a first semiconductor sensor and a second semiconductor sensor, according to certain embodiments;

FIGS. 5A-5B depict two schematic views of a non-limiting embodiment of a device comprising a first semiconductor sensor and a second semiconductor sensor;

FIGS. 6A-6E show exemplary illustrations of process steps to create a device, according to certain embodiments.

### DETAILED DESCRIPTION

Devices and methods for detection of biological agents (e.g., analytes) are described herein. The devices may be use an active detector sensor to detect analytes in a fluid in contact with the device. The analytes may bind to detector molecules which are functionalized on a surface of the sensor. Such binding can lead to a signal that may be measured to detect the presence of the analyte. As described further below, in addition to the active detector sensor, the device may utilize an reference (e.g., inactive) sensor which does not lead to a signal (or leads to a minimal signal compared to the signal of the detector sensor). The reference sensor may be positioned in close proximity to the detector sensor. The change in an electrical property (e.g., resistance) of the detector sensor as compared to the reference sensor may be measured. In this way, the reference sensor can provide a baseline measurement that is filtered out of the signal produced by the active detector sensor (e.g., using differential measurement techniques to remove noise that may exist in the sensor environment), allowing for more sensitive detection of the analyte. The sensors may be used to detect the presence of a specific molecule which may be useful for food safety, drug response, personalized medicine, cancer detection, disease verification, and other medical and biological applications.

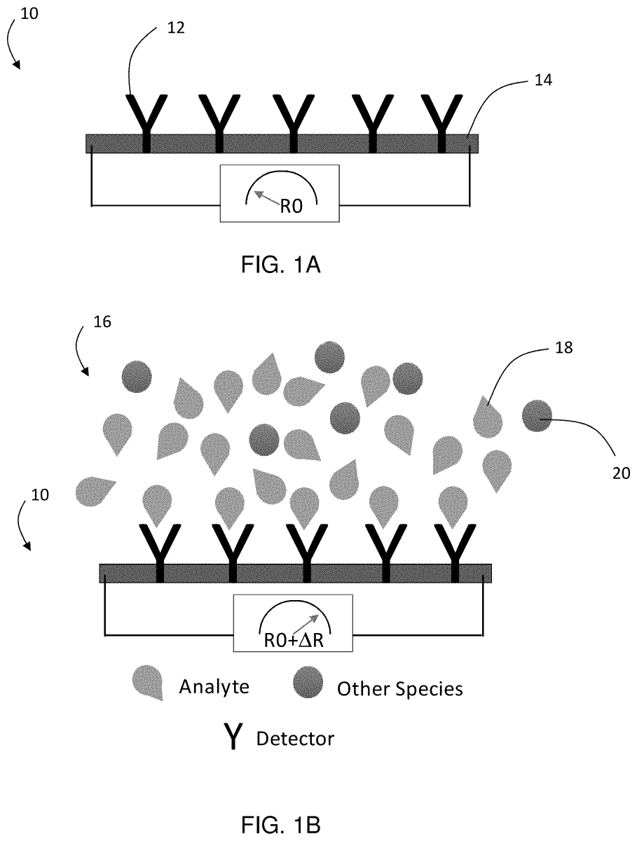

FIGS. 1A-1B show an active semiconductor-based detector sensor 10 according to certain embodiments. Detector molecules 12 are functionalized on a surface 14 of the sensor. As described further below, the detector molecules are selected for their ability to bind to an analyte that is desired to be detected. FIG. 1B shows a fluid 16 in contact with the sensor. As shown, the fluid includes analytes 18 and other species 20. In this embodiment, the analytes bind to the detector molecules. The binding of the analyte causes a measurable change in physical properties (e.g., electrical properties) of the semiconductor. For example, in the illustrative embodiment, a measured resistance change ΔR indicates the presence of the analyte, as illustrated in FIGS. 1A and 1B. In this manner, the sensor can be used to detect a certain analyte concentration in a fluid sample.

It should be understood that a change in physical properties different than resistance may be measured. For example, the change in conductance (or ΔG) may be measured in some embodiments; and, the change in conductivity may be measured in some embodiments. In some embodiments, structural changes in the detector molecule upon binding cause the measurable changes. In certain embodiments, the change is due to electrical gating by the analyte. In some embodiments, the change is due to a change in the surface plasmon resonance. In some embodiments, the change in property (e.g., conductance, resistance) may be generally detected electrically by applying an electric current to the sensor and measuring a change in voltage. In some embodiments, the change in property (e.g., conductance, resistance) may be generally detected electrically by applying an alternating electric field.

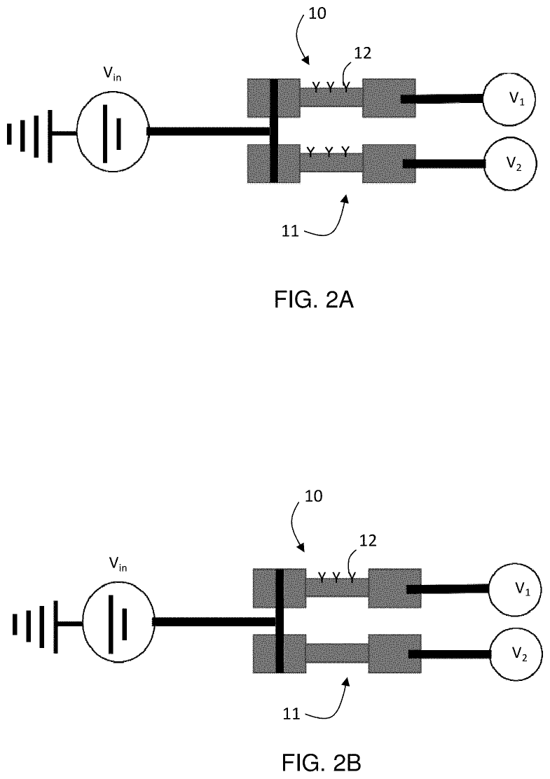

FIGS. 2A-2B show active semiconductor-based detector sensor 10 and a semiconductor-based detector reference (e.g., inactive) sensor 11. The reference sensor is positioned in close proximity to the detector sensor so that they are exposed to a similar environment.

As shown in FIG. 2A, in some embodiments, the reference sensor and the detector sensor both include detector molecules that can bind with analytes (not shown). In such embodiments, the binding of the detector molecules and analytes leads to a measurable change in properties (e.g., electrical properties) of the semiconductor of the detector sensor but does not lead to the same (or any) measurable change in properties of the semiconductor of the reference sensor.

As shown in FIG. 2B, in some embodiments, the detector sensor includes detector molecules that can bind with analytes (not shown) and the reference sensor does not include detector molecules. In such embodiments, the binding of the detector molecules and analytes leads to a measurable change in properties (e.g., electrical properties) of the semiconductor of the detector sensor but analytes are not bound to surface of the reference sensor so there is no measurable change in properties of the semiconductor of the reference sensor.

In both embodiments, the change in an electrical property (e.g., resistance) of the detector sensor as compared to the reference sensor can be measured. For example, the change in the electrical property of the semiconductor-based detector sensor is at least 2 times greater, in some cases at least 5 times greater, in some cases at least 10 times greater and in some cases at least 100 times greater than the change in a corresponding electrical property of the second semiconductor-based reference sensor. The reference sensor provides a baseline measurement that is filtered out of the signal produced by the active detector sensor (e.g., using differential measurement techniques to remove noise that may exist in the sensor environment), allowing for more sensitive detection of the analyte. Suitable differential measurement techniques have been described in commonly-owned U.S. patent application Ser. No. 14/510,178, filed Oct. 9, 2014, which is incorporated herein by reference in its entirety.

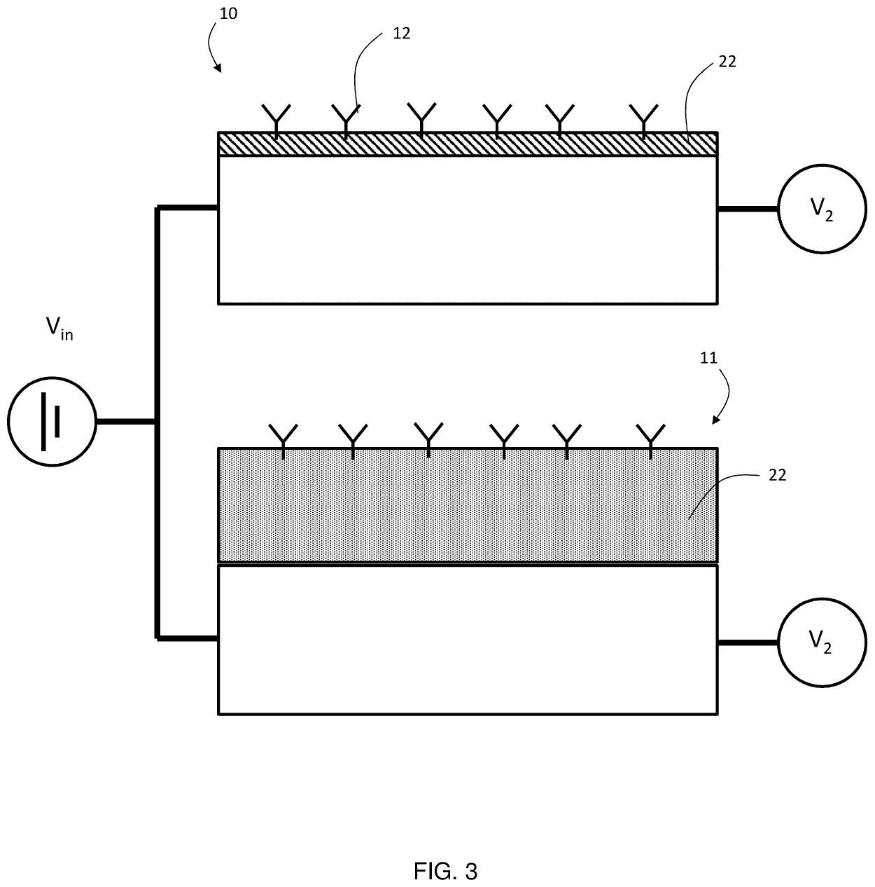

FIG. 3 shows active semiconductor-based detector sensor 10 and a semiconductor-based detector reference (e.g., inactive) sensor 11. Both the detector sensor and the reference sensor include an insulating layer 22. As shown, the insulating layer of the detector sensor is thinner than the insulating layer of the reference sensor.

The insulating layer of the reference sensor may be selected to be thick enough so that little or substantially no measurable change in properties of the semiconductor of the reference sensor occur when analytes (not shown) are bound to the detector molecules. For example, the thickness of the insulating layer of the reference sensor may be greater than or equal to 10 nm, greater than or equal to 25 nm, greater than or equal to 50 nm; and, in some embodiments, less than or equal to 100 nm, less than or equal to 50 nm, or less than or equal to 10 nm. It should be understood that any of the above-noted upper and lower limits may be suitable (e.g., greater than or equal to 10 nm and less than or equal to 100 nm).

In such embodiments, the insulating layer of the detector sensor may be selected to be thin enough so that a measurable change in properties of the semiconductor of the detector sensor occurs when analytes (not shown) are bound to the detector molecules. For example, the thickness of the insulating layer of the detector sensor may be greater than or equal to 2 nm, greater than or equal to 2 nm, greater than or equal to 4 nm; and, in some embodiments, less than or equal to 10 nm, less than or equal to 5 nm, or less than or equal to 4 nm. It should be understood that any of the above-noted upper and lower limits may be suitable (e.g., greater than or equal to 2 nm and less than or equal to 5 nm).

The thickness of the insulating layer of the reference sensor may be greater than or equal to 2 times, greater than or equal to 10 times or greater than or equal to 25 times the thickness of the insulating layer of the detector sensor. The thickness of the insulating layer of the detector sensor may be less than or equal to 50 times, less than or equal to 25 times or less than or equal to 10 times the thickness of the insulating layer of the reference sensor.

Any suitable insulating layers may be used including oxide (e.g., aluminum oxide, silicon oxide and the like) layers or nitride layers.

Other techniques may also be utilized to produce a reference sensor and the detector sensor. For example, the reference sensor may include a highly insulating layer and the detector sensor a less insulating layer of similar thickness to produce the signal differential. In some embodiments, the reference sensor is gated with an external voltage to further control its resistance.

In some embodiments, devices described herein may include the active sensor region, reference (e.g., inactive) sensor region, voltage input electrodes, voltage measurement electrodes and voltmeter. By way of example, each sensor region consists here of the functionalized nanowires plus pads upon which the electrodes connect. The pads provide a continuous pathway from the electrodes to the sensors.

In some embodiments, the two sensors are arranged parallel to each other. In some embodiments, the sensors are perpendicular. In certain embodiments, the sensors are arranged linearly. Other geometries may also be used for which the differential measurement is possible.

In the differential technique, a constant voltage Vin is applied to one side of both sensor areas and the resulting voltage difference across the other end of the sensors is measured. In some embodiments, a lock-in amplifier is employed to supply and measure the voltages. In some embodiments, an extra source-drain voltage may be applied to increase the sensitivity due to the non-ohmic nature of the semiconductor sensors (e.g., nanowires). Any universal noise, as well as any constant background signal, will be present at the same levels in both sensors' electrodes and will be systematically subtracted out, leaving only the resistance change of the active sensor relative to the reference sensor.

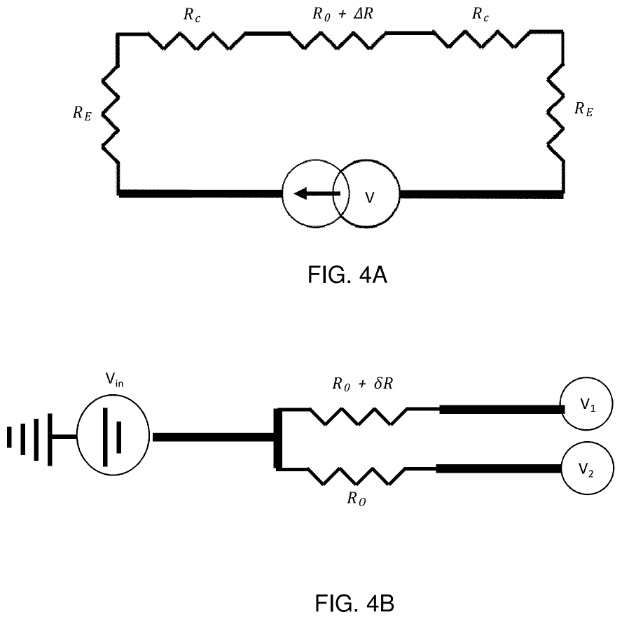

Referring to FIG. 4, the sensors can be considered as two parallel resistors. An input voltage Vin is applied to both sensors and the resulting voltage difference across the sensors is measured. R₀ refers to the bare resistance of the sensor, and ΔR is the change in resistance upon analyte binding. For demonstrational purposes, we assume here that R₀ is the same for the active and inactive sensor. The analysis is easily extended to the case where the bare resistances may be different, or where the inactive sensor's resistance can be gate-controlled. Noise appears as an additional δRᵢ for each sensor i, which is random in time, in each sensor, and noise δV_E,i which is random in time may appear in each separate post-sensor electrode labeled by i. Much of this noise, created by thermal fluctuations, fluid flows, and other large sources, is the same in each part of the circuit, so that δR₁=δR₂ and δV₁=δV₂ at all times. In some measurements, V₁ and V₂ are be inverted relative to their definitions here.

The measured voltage can be solved with simple circuit analysis. For the very small resistance changes expected for this invention,

ΔV=V₁−V₂=Vᵢₙ*δR/R₀

The small δR considered here was for demonstrational purposes. Larger resistance changes, which put V₁−V₂ into the regime that is nonlinear in δR, will be measurable with considerable reduced noise as well.

Any suitable semiconductor-based sensor device may be used for the sensors. For example, suitable devices have been described in commonly-owned U.S. patent application Ser. No. 14/510,178, filed Oct. 9, 2014 and commonly-owned International Application No. PCT/US2015/041527 which published as International Publication Number WO 2016/089453, all of which are incorporated herein by reference in their entireties.

The sensors comprise a semiconductor material. Suitable semiconductor materials from which a nanosensor can be made include, but are not limited to, silicon, germanium, III-V semiconductors, and the like. In some embodiments, the sensor comprises silicon. The sensor may include one or more layers formed on, or beneath, the semiconductor material such as additional semiconductor material layers or insulating layers (e.g., oxides such as silicon oxide).

In some embodiments, the semiconductor is patterned into nanowire(s). The sensor may be, in some embodiments, a field-effect transistor sensor (e.g., nanosensor). Generally, field effect transistors (FETs) use an electric field to control the electrical channel of conduction, and hence the conductivity of the charge carriers in the channel. The flow of charge carriers between the source and the drain can be tuned by modifying the size and the shape of the conducting channel by applying an electric field to the gate. In an exemplary biosensor configuration, the FET comprises a nanosensor (e.g., nanowire) channel between source and drain terminals. The nanosensor (e.g., nanowire) surface can be bio-functionalized so that a biomolecular binding event can create an electric field, similar to the control electric field applied to a conventional FET (FIG. 1). In certain devices that use the FET principle, a designated, physically separated sensor surface can be formed by precision manufacturing. The FET sensor can be connected to an electronic circuit to monitor the specific conductance of the sensor surface. In some embodiments, operationally, many independent electronic circuits may be interrogated in a massively parallel manner. FET biosensors can be adapted for the measurement of biomolecules interacting with such a sensor surface (FIGS. 1A and 1B).

In some embodiments, the sensor comprises a nanoscale silicon-based FET device.

Many such devices show sensitivity, reliability, robustness and the sensor flexibility needed for many multiplexed diagnostics microarrays. In some cases, the nanoscale devices can be developed and/or implemented on traditional top-down silicon. In some cases, by developing and implementing the nanoscale devices on traditional top-down silicon, the reliability and robust quality of top-down silicon semiconductor manufacturing processes can be improved and error rates in testing, both in point-of-care and central reference labs can be reduced. This can, in some cases, result in increased effectiveness of each patient visit to a lab or clinic, reduced cost of diagnosis, and earlier diagnosis, treatment, and monitoring.

In one particular embodiments, the nanosensor is a silicon nanochannel field effect transistor (FET) biosensor. Such sensors can be used to perform highly sensitive and/or label-free analyte detection. Such sensors can have exceptional electrical properties and small dimensions. In certain embodiments, the silicon nanochannels are ideally suited for extraordinarily high sensitivity. In some cases, the high surface-to-volume ratios of these systems make single molecule detection possible. In some cases, such FET sensors (e.g., biosensors) offer the benefits of high speed, low cost, and high yield manufacturing, without sacrificing the sensitivity typical for traditional optical methods in diagnostics. Top down manufacturing methods can be used to leverage advantages in Complementary Metal Oxide Semiconductor (CMOS) technologies, allowing for richly multiplexed sensor arrays. Examples of nanochannel based sensor systems are described, for example, in International Patent Publication WO 2008/063901A1 by Yu Chen et al., and International Patent Publication WO 2009/124111A1 to Mohanty et al., each of which is incorporated by reference in its entirety for all purposes.

In some embodiments, the semiconductor-based sensor can be part of a bias and measurement circuit. In some embodiments, the bias and measurement circuit is operated by applying a bias voltage across two ends of the nanosensor (e.g., nanochannels) within the circuit. The bias voltage can be selected to be sufficiently negative to achieve a desired dependence of the differential conductance of the sensing element on the surface potential of the sensor (e.g., of the nanochannels). In certain embodiments, this dependence has a steeply sloped region of high amplification which is substantially greater than a reference amplification exhibited by the sensing element at a zero-bias condition, thus achieving relatively high signal-to-noise ratio. The bias and measurement circuit measures, in some embodiments, the differential conductance of the sensing element and converts the measured differential conductance into a signal indicative of presence or activity of the analyte. In certain embodiments, the measured differential conductance can be converted into a signal indicative of the presence or activity of the analyte by using a look-up table or alternative conversion mechanism reflecting a prior calibration operation. In some embodiments, applied gate voltage can be used to control a sensor's sensitivity. The bias and reference gate voltage can be used independently, according to certain embodiments, to control sensitivity.

As described above, at least a portion of the sensor (e.g., a surface) is functionalized with a detector molecule. The detector molecule may comprise, for example, an antibody, enzyme, protein, peptide, small molecule, nucleic acid, aptamer, receptor molecule, polymer, and/or a supramolecular structure. The detector molecules may be designed to be particle-specific, i.e. only one specific analyte will bind to a given detector molecule. In some embodiments, the detector molecule is an antibody. In certain embodiments, the detector molecule is or comprises a DNA or RNA fragment. In some embodiments, the detector molecule is or comprises glucose oxidase. In some cases, the detector molecule is or comprises urease.

The analyte may comprise, for example, a protein, a small molecule, a nucleic acid, a peptide, an antibody, an aptamer, a biomarker, a gene, a virus particle, a supramolecular structure, a macromolecule, a receptor molecule, a biological cell, and/or a biological cell cluster.

In some cases, the analyte comprises a biomolecule. In some instances, the analyte may be a protein biomarker or a gene biomarker. In certain embodiments, the analyte is or comprises a monosaccharide and/or a polysaccharide. For example, in some cases, the analyte is or comprises glucose. One non-limiting example of a small molecule analyte is urea.

It should be understood that the methods described herein are not limited to any particular detector molecule or analyte.

In some embodiments, the analyte and the detector molecule may associate via a chemical interaction, such as a chemical bond. The chemical bond may be a covalent bond or non-covalent bond. In some cases, the chemical bond is a non-covalent bond such as a hydrogen bond, ionic bond, dative bond, and/or a Van der Waals interaction. One or more of the species and/or agents (e.g., analyte, detector molecules) may comprise functional groups capable of forming such bonds. It should be understood that covalent and non-covalent bonds between components may be formed by any type of reactions, as known to those of ordinary skill in the art, using the appropriate functional groups to undergo such reactions. Chemical interactions suitable for use with various embodiments described herein can be selected readily by those of ordinary skill in the art, based upon the description herein.

In some embodiments, an association between the analyte and the detector species may occur via a biological binding event (i.e., between complementary pairs of biological molecules). For example, the analyte or the detector molecule may include an entity such as biotin that specifically binds to a complementary entity, such as avidin or streptavidin, on another species or agent. Other examples of biological molecules that may form biological bonds between pairs of biological molecules include, but are not limited to, proteins, nucleic acids, glycoproteins, carbohydrates, hormones, and the like. Non-limiting examples include, but are not limited to, an antibody/peptide pair, an antibody/antigen pair, an enzyme/substrate pair, an enzyme/inhibitor pair, an enzyme/cofactor pair, a protein/substrate pair, a nucleic acid/nucleic acid pair, a protein/nucleic acid pair, a peptide/peptide pair, a protein/protein pair, a small molecule/protein pair, a receptor/hormone pair, a receptor/effector pair, a ligand/cellular receptor pair, a biotin/avidin pair, a biotin/streptavidin pair, a drug/target pair, small molecule/peptide pair, a small molecule/protein pair, and a small molecule/enzyme pair. Biological interactions between species and/or agent(s) for use in the embodiments described herein can be selected readily, by those of ordinary skill in the art, based upon the description herein as their function, examples of such biological interactions, and knowledge herein and in the art as to simple techniques for identifying suitable biological interactions.

In certain embodiments, the analyte and the detector species may be associated with each other via a physical interaction. For example, in some embodiments, analyte (e.g., supramolecular structure) may be physically entangled with at least a portion of the detector species (e.g., macromolecule).

In certain embodiments, the analyte and the detector species may be associated with each other via a linking moiety (e.g., other biological or chemical species that causes the analyte and the detector species to be in close proximity. For example, the shortest distance between the analyte and the detector species associated with each other may be greater than a Debye length. In some instances, the shortest distance may be less than or equal to about 100 nanometers, less than or equal to about 50 nanometers, less than or equal to about 25 nanometers, less than or qual to about 10 nanometers, or less than or equal to about 1 nanometers.

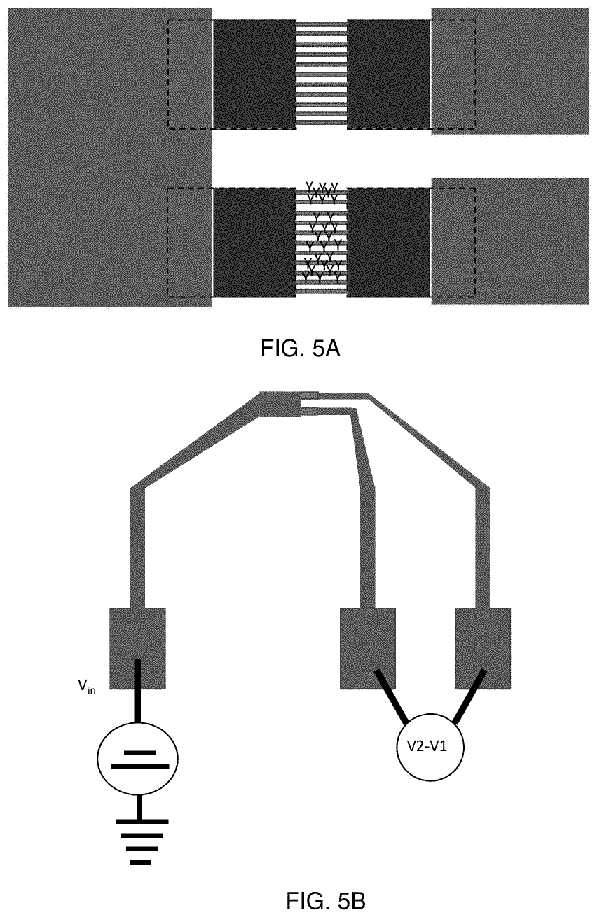

FIGS. 5A and 5B show one embodiment of the differential resistance biosensor integrated onto a microchip. A close-up of the sensor region shown in FIG. 5B, comprising: the active sensor (semiconductor nanowires connected to semiconductor pads); the inactive sensor, which is inactivated by not functionalizing; and the voltage output electrodes 4. In some embodiments, the electrodes are coated with an insulating layer, including but not limited to, Al₂O₃, SiO₂, HfO₂, or Si₃N₄. FIG. 5 shows the complete microchip, including bonding pads that connect to external circuitry. In this embodiment, the sensors are designed parallel to each other with the nanowires parallel to the electrodes. Some embodiments will have different bonding pad configurations and different electrode configurations.

### Example

This is a non-limiting example of process by which a representative sensor is made including drawings of example geometries. The sensor is made from a thin silicon layer, typically around 100 nm thick, which is on top of a silicon dioxide layer, typically 200 nm thick or greater.

In some embodiments, ions are implanted into the silicon in the region where the connection pads are to be made. The ions can be metals or other dopants, as mentioned previously.

Next, the nanowires and electrode attachment pads are created by electron beam lithography and reactive ion etching, which removes silicon everywhere except the sensor region. In some embodiments, defining the nanowires and pads is split into two steps, whereby the nanowires and pads are defined separately. In some embodiments, the nanowires are defined with electron beam lithography and the pads with photolithography. In some embodiments, the pads and nanowires are all defined with photolithography. Our invention generally covers all methods of creating the nanowires and pads.

Then the pattern for the metal electrodes in the 4-point measurement geometry is created with photolithography and the metal electrodes are deposited. The electrodes comprise a metal, such as Au, Cu, Ag, Al, and may be an alloy or metallic multilayer. Often an adhesion layer of Ti, Ta, or another metal will be used. The electrodes are approximately 10 to 20 microns wide near the sensor and become wider farther away. Typically, the metal thickness is in the range of 100 nm.

The electrodes are next coated with a thick insulating barrier, typically about 100 nm, typically an oxide such as Al₂O₃, SiO₂, HfO₂, or Si₃N₄. The thick barrier also covers one of the sensors, causing it to be deactivated.

The sensors and electrodes are then coated with a top thin insulating barrier, typically 10 nm, of a similar oxide.

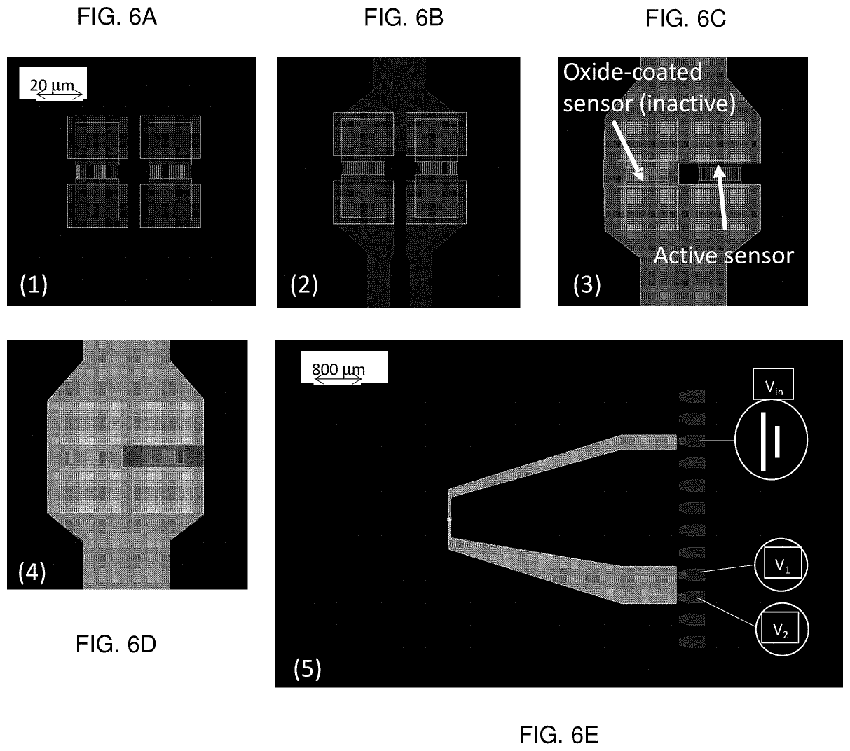

Finally, metallic pads are deposited to allow connection to external measurement equipment. The pads are typically 1 to 2 microns thick highly conductive noble metal, such as Au, Ag, Cu. They typically include an adhesion layer such as thin (10 nm thick) Ti. The final metal deposition step may also involve deposition of extra electrodes for further sensor enhancements. A schematic of the finished sensor, ready for integration into the final circuit, is shown in FIG. 6.

What is claimed is:

1. A device, comprising
a first semiconductor-based sensor comprising a first insulating layer functionalized with a detector molecule; and
a second semiconductor-based sensor in electrical communication with the first semiconductor-based sensor, the second semiconductor-based sensor comprising a second insulating layer,
wherein the first insulating layer and the second insulating layer are configured such that when the first semiconductor-based sensor and the second semiconductor-based sensor are exposed to a fluid comprising an analyte, the change in an electrical property of the first semiconductor-based sensor is greater than the change in a corresponding electrical property of the second semiconductor-based sensor.

2. A device, comprising
a first semiconductor-based sensor comprising a first insulating layer functionalized with a detector molecule; and
a second semiconductor-based sensor in electrical communication with the first semiconductor-based sensor, the second semiconductor-based sensor comprising a second insulating layer;
wherein the first insulating layer is at least 2 times the thickness of the second insulating layer.

3. The device of claim 1, wherein the second insulating layer is functionalized with the detector molecule.

4. The device of claim 1, wherein the change in the electrical property of the first semiconductor-based sensor is at least two times greater than the change in a corresponding electrical property of the second semiconductor-based sensor.

5. The device of claim 1, wherein the capacitance between the first semiconductor-based sensor and a layer of charge formed when the first semiconductor-based sensor is exposed to the fluid and the analyte binds the detector molecule of the first semiconductor-based sensor is greater than the capacitance between the second semiconductor and a layer of charged formed when the second semiconductor-based sensor is exposed to the fluid and the analyte binds the detector molecule of the second semiconductor-based sensor.

6. The device of claim 1, wherein the thickness of the second insulating layer is greater than or equal to 2 times the thickness of the first insulating layer and less and less than or equal to 50 times the thickness of the first insulating layer.

7. The device of claim 1, wherein the thickness of the first insulating layer is greater than or equal to 2 nm and less than or equal to 5 nm.

8. The device of claim 1, wherein the thickness of the second insulating layer is greater than or equal to 10 nm and less than or equal to 100 nm.

9. The device of claim 1, wherein the dielectric constant of the first insulating layer is greater than the dielectric constant of the second insulating layer.

10. The device of claim 1, wherein the first insulating layer and/or the second insulating layer comprises an oxide.

11. The device of claim 1, wherein the detector molecule comprises an antibody, a DNA fragment, and/or RNA fragment.

12. The device of claim 1, wherein the detector molecule comprises an enzyme.

13. The device of claim 1, wherein the detector molecule comprises glucose oxidase.

14. The device of claim 1, wherein the detector molecule comprises urease.

15. The device of claim 1, wherein the analyte comprises a biomolecule.

16. The device of claim 1, wherein the analyte comprises a monosaccharide and/or a polysaccharide.

17. The device of claim 1, wherein the analyte comprises glucose.

18. The device of claim 1, wherein the analyte comprises urea.

19. The device of claim 1, wherein the electrical property is an electrical property selected from the group consisting of voltage, resistance, conductance, and current.

20-23. (canceled)

24. A method, comprising:
exposing a device to a fluid comprising an analyte, the device comprising:
a first semiconductor-based sensor comprising a first insulating layer functionalized with a detector molecule, and
a second semiconductor-based sensor in electrical communication with the first semiconductor-based sensor, the second semiconductor-based sensor comprising a second insulating layer; and
measuring a change in an electrical property of the first semiconductor-based sensor and a change in a corresponding electrical property of the second semiconductor-based sensor to determine a differential electrical property based at least in part on the change in the electrical property of the first semiconductor-based sensor and the change in the electrical property of the second semiconductor-based sensor.

25-45. (canceled)
